(19) United States
(12) Patent Application Publication
(10) Pub. No.: US 2015/0032093 A1
(43) Pub. Date: Jan. 29, 2015

(54) THERMAL TISSUE CLOSURE DEVICE WITH TEMPERATURE FEEDBACK CONTROL

(71) Applicant: Calore Medical Ltd., Or Akiva (IL)

(72) Inventors: Giora Kornblau, Binyamina (IL); David Maier Neustadter, Moshav Nof Ayalon (IL)

(21) Appl. No.: 14/339,610

(22) Filed: Jul. 24, 2014

Related U.S. Application Data
(60) Provisional application No. 61/858,148, filed on Jul. 25, 2013.
(60) Provisional application No. 62/015,968, filed on Jun. 23, 2014.

(51) Int. Cl. A61B 18/08

(52) U.S. Cl. CPC A61B 18/082; A61B 2018/00714
USPC 606/31

(57) ABSTRACT

A device and a method for shrinkage of tissue containing collagen is described. The device comprises a heating tip, a support shaft, and a control system. The heating tip comprises a heating element and a temperature sensor therein. The control system is configured to receive a temperature feedback from the temperature sensor and to regulate energy delivered to the heating element based on the temperature feedback. Heating of the tissue region causes shrinkage of collagen-containing tissue, thereby closing any perforation or opening in the tissue region.

This application claims priority to U.S. Provisional Application No. 61/858,148, filed Jul. 25, 2013, and U.S. Provisional Application No. 62/015,968 filed Jun. 23, 2014, which are incorporated herein by reference in their entirety.

The present disclosure is directed towards a device for controlled heating of tissue containing collagen to cause tissue shrinkage, and more specifically, a device for controlled heating to close a perforation or a puncture in a tissue region containing collagen.

### BACKGROUND

Heat may be used to shrink tissue containing collagen in various surgical and diagnostic procedures. For example, heat may be used to close and/or heal perforations or openings in tissue walls. For example, heat may be used to close arteriotomies on blood vessel walls by denaturing collagen within the tissue, thereby shrinking the tissue around the perforation/opening, and/or by inducing blood coagulation. In such procedures, if the temperature of the tissue reaches a temperature that exceeds a threshold, damage may be caused to the tissue. Thus, if a system were to have an open loop design without sufficient temperature control, effectiveness might be sacrificed, or harm to the patient's body might be caused.

### SUMMARY

Some aspects of the present disclosure may involve devices, systems, and methods where temperature is regulated during tissue shrinkage. Thus, for example, temperature of a target tissue region, and/or the temperature of a distal region of a closure device, may be monitored while heat is applied to the tissue. Adjustments may be made to maintain the tissue temperature, and/or the temperature of the distal region of the closure device, at a predefined level.

One application of the devices and methods of the present disclosure is the thermal closure of puncture sites (arteriotomies) on blood vessel walls. The particular requirements of small size, vessel contact area, the need for a vessel anchor which passes through the heating element, etc., may, in some circumstances, make incorporation of temperature feedback into vascular closure devices particularly challenging. The present disclosure describes devices and methods that address the potential challenges of a closed loop system for vascular tissue closure. The application of the present disclosure is not limited to the blood vasculature, and may be applied to any vessel, duct, cavity, and/or tissue tract found in the body.

One aspect of the present disclosure may include a device for controlled shrinkage of a tissue region containing collagen. The device may include a heating element configured to deliver heat to the tissue region, a support for positioning the heating element at a location to effect delivery of heat to the tissue region, a temperature sensor associated with the heating element, and a control system comprising a controller/processor configured to access information related to tissue shrinkage and to regulate the temperature of the heating element.

Another aspect of the present disclosure may include a method of closing a perforation in a tissue region containing collagen, the method including the steps of providing a tissue closure device comprising a heating Up having a heating element and one or more temperature sensors, and at least one processor for regulating energy transmission from a power source to the heating element. The method may further include the steps of advancing the heating tip percutaneously to the perforation in the tissue region, delivering energy to the heating element to heat the tissue region and cause shrinkage of the collagen-containing tissue surrounding the perforation, monitoring a temperature of at least one of the heating tip and the target tissue region using the one or more temperature sensors, and regulating energy delivered to heating element based on the temperature of the heating tip and/or the temperature of the target tissue region.

Other aspects of this disclosure are contained in the accompanying drawings, description, and claims. Thus, this summary is exemplary only, and is not to be considered restrictive.

The accompanying drawings, which are incorporated in and constitute a part of this specification, illustrate embodiments of the invention and together with the description, serve to explain the principles of the various aspects of the invention.

### BRIEF DESCRIPTION OF DRAWINGS

FIG. 1 illustrates a tissue closure device, in accordance with exemplary embodiments of the present disclosure;

FIG. 2 illustrates another tissue closure device, in accordance with exemplary embodiments of the present disclosure; and

FIG. 3 shows related art data about the amount of time required to achieve tissue shrinkage as a function of tissue temperature.

It is to be understood that both the foregoing general description and the following detailed description are exemplary and explanatory only and are not restrictive of the invention, as claimed.

### DESCRIPTION OF EXEMPLARY EMBODIMENTS

Reference will now be made to certain embodiments consistent with the present disclosure, examples of which are illustrated in the accompanying drawings. Wherever possible, the same reference numbers are used throughout the drawings to refer to the same or like parts. It is to be understood that the device of the present disclosure can be employed to close or heal perforations in any tissue region of a patient's body, or to generally cause shrinkage of tissue in any part of a patient's body.

The present disclosure describes a tissue closure device and a method for controlled shrinkage of tissue containing collagen. Exemplary embodiments may include a heating element configured to deliver heat to a tissue region containing collagen. The heating element may include any electrical, chemical, mechanical, or other mechanism for causing heat, such as, for example, ultrasound heating, RF heating, laser heating, microwave heating, or any device capable of converting electricity into heat through a process of resistive or Joule heating. In such a device, when electric current passes through a resistive element, heating of the element occurs. The element may be made of any material capable of resulting in heat, including metal, ceramic, composites, or combinations thereof. Similarly, the heating element may be structured in any way so long as it is capable of causing the desired heating. Exemplary shapes include tubes, wires, films, foils, coils, and any other shape or construction capable of producing the desired heat. The heating element may be encased in a heat-conductive housing or may be otherwise provided to enable the resulting heat to be directed to a target tissue area.

Disclosed embodiments may also include a support for positioning the heating element at a location to effect delivery of heat to the tissue region. The support may include any structure that enables positioning of the heating element adjacent target tissue. Thus, depending on the target tissue at issue, the support may assume alternative shapes or configurations. By way of example, the support may be an elongate element, The particular shape and construction of an elongated support may vary, and might include, by way of example only, one or ore of a tube, rod, shaft, bar, rib, or column.

The support may be configured to position the heating element. Such a configuration may include an opening in the shaft in which the heating element is housed, or a connection on the shaft that affixes the heating element to the shaft, Such a connection might be permanent or might enable the heating element to be removed from the shaft. Alternatively, the heating element or a housing of the heating element may be integrated into the shaft, or may be integrally formed with the shaft. Regardless, each of the foregoing are examples of supports configured to position the heating element.

Embodiments of the invention may also include a temperature sensor associated with the heating element. As used herein, the term “associated with” includes any relationship where heat generated via the heating element may be detected, regardless of whether the temperature of the heating element is itself detected, whether the temperature of some other structure is measured, or whether a temperature of surrounding tissue is detected.

The temperature sensor itself may include any structure capable of either detecting or measuring temperature, inducing, for example, a thermometer, bimetal, thermocouple, resistance thermometer, silicon bandgap temperature sensor, or any other arrangement or structure capable of providing feedback indicative of temperature.

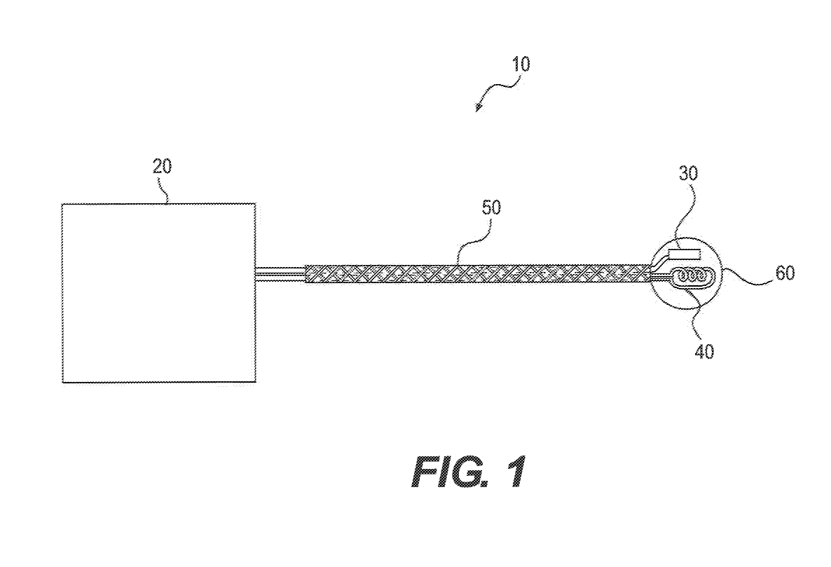

FIG. 1 shows an exemplary embodiment of a tissue closure device 10 comprising a control system 20, a heating element 40, a temperature sensor 30, and a support shaft 50 connecting control system 20 to temperature sensor 30 and heating element 40. Heating element 40 and/or temperature sensor 30 may be located within a heating tip 60. The size and configuration of heating tip 60 may be selected to facilitate thermal contact between heating tip 60 and the target tissue region, and enhance heat conduction between heating element 40 and the target tissue region. By way of example only, heating tip 60 may be made of a material having heat conductance greater than 100 W/(mK), or any other conductance capable of conveying heat in a quantity to effect shrinkage of collagenous tissue, and thereby close or otherwise promote healing of a perforation or an opening in the tissue region.

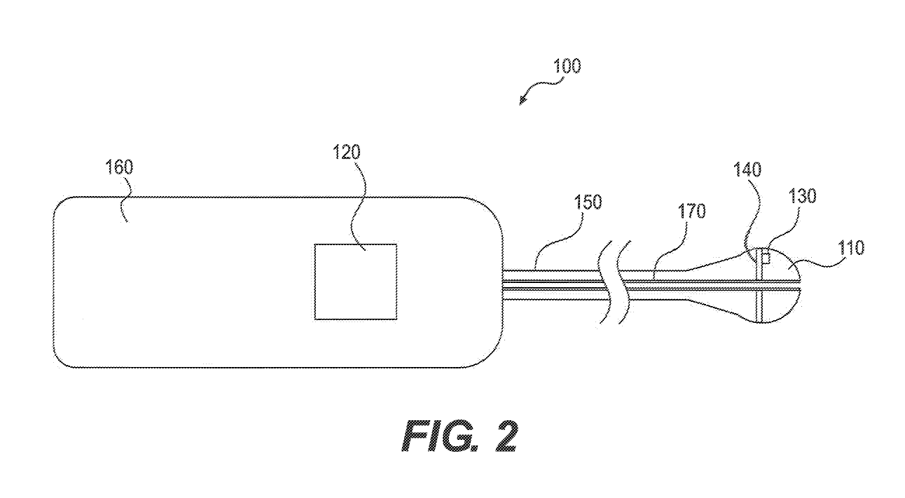

In exemplary embodiments, heating tip 60 may have a spherical or semi-spherical shape. In other embodiments, heating tip 110 may be dome-shaped, as is shown in FIG. 2. In an exemplary embodiment, heating tip 60 may have a diameter of about 3 mm to about 6 mm.

Although FIG. 1 illustrates a single temperature sensor 30, some embodiments may include multiple temperature sensors 30 associated with heating element 40. In such embodiments, one or more temperature sensors 30 may be utilized to determine the temperature of heating element 40 or other physical structure, and/or one or more temperature sensors 30 may be employed to determine the temperature of the target tissue region.

The location of temperature sensor 30 within heating tip 60 may be determined based on whether temperature sensor 30 is configured to monitor the temperature of the tissue region, the temperature of heating tip 60, or the temperature of some other component that correlates to the amount of heat received by the tissue.

Embodiments of the invention may also include at least one processor, configured to access information related to tissue shrinkage and to regulate the temperature of the heating element. As used herein, the term “processor” may include an electric circuit that performs a logic operation on input or inputs. For example, such a processor may include one or more integrated circuits, microchips, microcontrollers, microprocessors, all or part of a central processing unit (CPU), graphics processing unit (GPU), digital signal processors (DSP), field-programmable gate array (FPGA) or other circuit suitable for executing instructions or performing logic operations. The at least one processor, may be included in or may be coincident with, for example, control system 20, schematically depicted in FIG. 1. Control system 20 may be configured to regulate an associated power source within (or external to) control system 20, to enable selective delivery of energy to heating element 40. In one embodiment, heating element 40 may receive energy from the power source via an electrical conductor that runs through support 50. The power source may provide electrical energy to heating element 40 to heat the tissue region, and thereby effect shrinkage of collagen-containing tissue to close or heal a perforation or an opening in the tissue region.

As discussed later in greater detail, the information related to tissue shrinkage may include a target temperature, a number of target temperatures or a target temperature profile (time-temperature). The accessed information may be stored within the processor itself or may be stored in memory accessed by the processor.

In exemplary embodiments, control system 20 may include a closed loop system, coupling temperature sensor 30 to the power source. In such a system, the temperature of the tissue region or heating element 40 may be monitored, and the power delivery to heating element 40 may be adjusted to follow a predefined heating profile (discussed in detail below). In some embodiments, a physician operating tissue closure device 10 may be permitted to override the closed loop system, if desired.

In an exemplary embodiment, the at least one processor may monitor the temperature and adjust the power delivered to heating element 40. The at least one processor may be configured to switch power on and off, and/or to modulate the power delivered to heating element 40.

FIG. 2 shows an exemplary embodiment of a tissue closure device 100, which may operate using the same or differing principles described in connection with FIG. 1. Tissue closure device 100 may include a handle 160, a heating tip 110, and an elongate shaft 150 connecting handle 160 to heating tip 110.

In exemplary embodiments, tissue closure device 100 may be configured for use in conjunction with a guidewire, which assists in guiding and placing tissue closure device 100 at the target region. In such embodiments, elongate shaft 150 as well as heating tip 110 may include a lumen with an opening 170, as shown in FIG. 2, allowing for placement of the device over a guidewire.

In exemplary embodiments, control system 120 may be housed within handle 160. In some such embodiments, control system 120 includes a power source, which may be housed within the handle as part of the control system itself, or as a separate component. The power source may be configured for electrical connection to a heating element 140 provided in heating tip 110. In exemplary embodiments, the power source may also be electrically connected to a temperature sensor 130 provided in heating tip 110. The temperature of the target tissue region, or the temperature of heating element 140, or the temperature of the heating tip 110, may be monitored by temperature sensor 130, and the power delivered to heating element 140 may be adjusted to maintain a preselected thermal profile (discussed in detail below). A microprocessor or a controller may be included and incorporated into control system 120 to modify the power delivered to heating element 140 based on the temperature information received by the controller or the microprocessor from temperature sensor 130.

In exemplary embodiments, the power source of tissue closure device 100 may include one or more batteries. In such embodiments, the power source may be located within handle 160. In some embodiments, handle 160 further include indicators that signal the various steps of the procedure to a physician using the device.

Exemplary embodiments of the present disclosure may be utilized to close and provide hemostasis of a puncture site in a body lumen, particularly blood vessels of the human body. In some such embodiments, an anchor device may be introduced into the vessel and used to temporarily occlude the puncture site. The tissue closure device 100 may then be introduced into the body over the shaft of the anchor device such that the anchor shaft goes through the lumen of shaft 150 and heating tip 110. The anchor device may continue to occlude the puncture site while heating tip 110 may be used to shrink the collagenous tissue around the puncture site. The anchor device may be removed prior to complete closure/occlusion of the puncture as a result of the shrinkage of collagen-containing tissue.

The present disclosure also describes a method of shrinking tissue containing collagen by applying heat, but without causing damage or destruction of either the target or surrounding tissues. In exemplary embodiments, the heat delivery system is a tissue closure device, such as, tissue closure device 10/100. In such embodiments, tissue closure device 10/100 may apply heat to the target region in accordance with a thermal profile that is based on a recognized property of thermal shrinkage of collagen-containing tissue when exposed to temperatures above normal body temperature.

In exemplary embodiments, the temperature of the target tissue region is elevated to about 60° C. to about 110° C. In one such embodiment, the temperature of the target tissue region is elevated to about 70° C. and maintained at that temperature for a set period of time, At 100° C., the water in the blood and tissue begins to boil which may interfere with heat transfer and/or release gases within the treatment area. Therefore, to minimize tissue damage, in some embodiments the tissue temperature might be controlled so as not to exceed 100° C. In some embodiments, the controller/processor of tissue closure device 10/100 is configured to turn off heating element 40/140 automatically if the sensed temperature of the tissue region or the sensed temperature of heating element 40/heating tip 110 approaches 100° C. In other embodiments, the controller/processor of tissue closure device 10/100 is configured to automatically disconnect heating element 40/140 from the power source when a target temperature of the tissue region and/or the target temperature of heating element 40/heating tip 110 is reached.

In exemplary embodiments, heating element 40/heating tip 110 is raised to a temperature above the desired temperature of the target tissue region in order to account for the temperature differential between heating element 40/heating tip 110 and the target region. In one such embodiment, heating tip 110 is heated to a temperature of 95° C. in order to heat the target tissue region to about 70° C.

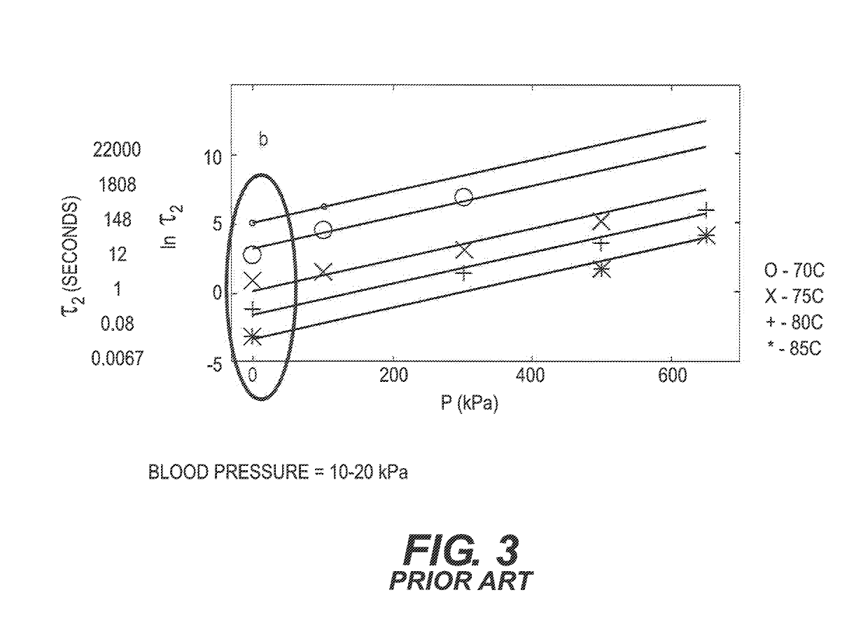

In exemplary embodiments, tissue closure device 10/100 applies heat to the target region in accordance with a thermal profile that is based on the temperature of the heating element 40/heating tip 110 and a set heating time. As shown in prior art FIG. 3, the amount of time required to shrink collagenous tissue to a desired level decreases as temperature is increased. (See N. T. Wright and J. D. Humphrey, Denaturation of Collagen via Heating: An Irreversible Rate Process, Annu. Rev. Biomed. Eng. (2002) 4:109-28 at p. 116.) The controller/processor of control system 20/120 regulates power (i.e., current and/or voltage and/or pulse width) to heating element 40/140 based on the thermal profile. The temperature feedback received from temperature sensor 30/130 allows the controller to maintain the temperature of heating element 40/heating tip 110 at the set temperature of the selected thermal profile independent of the thermal properties of the surrounding tissues.

In exemplary embodiments, a predefined heating time of about 5 seconds to about 20 seconds is used. In some embodiments, the predefined heating time is about 10 seconds. In some other embodiments, the predefined heating time is about 5 seconds to about 20 seconds and the temperature of the heating tip 110 is set at about 95° C. In one such embodiment, the predefined heating time is about 10 seconds and the temperature of heating tip 110 is set at about 95° C. in order to heat the target tissue region to about 70° C. Thermal simulation of heat treatment of blood vessel walls have shown that if the temperature of heating tip 110 is set at about 95° C. (and assuming temperature of blood flowing through the vessels is 37° C.), the target region of the vessel wall will be heated to about 70° C. to a depth of about 0.3 mm.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A device for controlled shrinkage of a tissue region containing collagen, the device comprising:
a heating element configured to deliver heat to the tissue region;
a support for positioning the heating element at a location to effect delivery of heat to the tissue region;
a temperature sensor associated with the heating element; and
at least one controller configured to access information related to tissue shrinkage and to regulate the temperature of the heating element.

2. The device of claim 1, wherein the support is configured to direct the heating element towards a perforation in the tissue region.

3. The device of claim 1, further comprising a heating tip configured to contact the tissue region, and wherein the heating element and the temperature sensor are located within the heating tip.

4. The device of claim 3, wherein the heating tip is connected to the support, and wherein the support is configured to direct the heating tip towards a perforation in the tissue region.

5. The device of claim 3, wherein the heating tip is shaped as a dome.

6. The device of claim 3, wherein the heating tip and the support include a lumen therethrough for placement over a guide wire.

7. The device of claim 3, wherein the information related to tissue shrinkage is at least one of a temperature of the heating tip and a temperature of the tissue region.

8. The device of claim 3, wherein the heating tip has a diameter of about 3 mm to about 6 mm.

9. The device of claim 3, wherein the temperature sensor associated with the heating element is arranged in a manner to permit sensing a temperature of at least one of the heating tip and the tissue region.

10. The device of claim 1, wherein the controller is configured to receive temperature feedback from the temperature sensor and to regulate energy delivered to the heating element based on the temperature feedback.

11. A method of closing a perforation in a tissue region containing collagen, comprising:
providing a tissue closure device comprising a heating tip having a heating element and at least one temperature sensor, and at least one processor for regulating energy transmission from a power source to the heating element;
advancing the heating tip percutaneously to the perforation in the tissue region;
delivering energy from the power source to the heating element to heat the tissue region and cause shrinkage of the collagen-containing tissue surrounding the perforation;
monitoring a temperature of at least one of the heating tip and the tissue region using the at least one temperature sensors; and
regulating energy delivered to the heating element based on at least one of the temperature of the heating tip and the temperature of the tissue region.

12. The method of claim 11, further comprising maintaining a temperature of the tissue region between about 70° C. to about 110° C.

13. The method of claim 11, further comprising automatically disconnecting the heating element from the power source when a target temperature of the tissue region and/or the heating tip is reached.

14. The method of claim 11, further comprising maintaining a target temperature of the tissue region and/or the heating tip for about 10 seconds.

15. The method of claim 14, further comprising maintaining a temperature of the tissue region at about 70° C. for about 10 seconds.

16. The method of claim 15, further comprising maintaining a temperature of the tissue region at about 70° C. by maintaining a temperature of the heating tip at about 95° C.

17. The method of claim 11, further comprising regulating a duration of heating based on feedback from the temperature sensor.

18. The method of claim 11, further comprising preventing a temperature of the tissue region from exceeding approximately 100° C.

19. The method of claim 11, further comprising maintaining a temperature of the heating tip at about 95° C. for a duration of about 5 seconds to about 20 seconds.

20. The method of claim 19, further comprising maintaining a temperature of the heating tip at about 95° C. for a duration of about 10 seconds.
